(19) United States
(12) Patent Application Publication
(10) Pub. No.: US 2015/0051627 A1
(43) Pub. Date: Feb. 19, 2015

(54) Centering Scalpel

(71) Applicant: Frederick George, Christchurch (NZ)

(72) Inventor: Frederick George, Christchurch (NZ)

(21) Appl. No.: 13/969,488

(22) Filed: Aug. 16, 2013

(51) Int. Cl. A61B 17/3211

(52) U.S. Cl. CPC A61B 17/3211
USPC 606/167

(57) ABSTRACT

A scalpel blade for splitting toenails that has a sharpened end in a concave shape that self centers on the thin nail edge, slicing the nail plate from above and below simultaneously, making the nail splitting operation safer and easier.

### TECHNICAL FIELD

This invention is concerned with improving the performance of scalpels particularly useful in toenail surgery.

### BACKGROUND ART

Medical scalpels designed for toenail surgery, particularly scalpels designed to split the side of the toenail longitudinally away from the main part of the nail in ingrown toenail surgery have heretofore been thin, elongated blades with the narrow tip of the blade sharpened into a squared off chisel shape.

This chisel blade requires significant training and manual dexterity to successfully split the length of the toenail without slipping above or below the nail plate or deviating from the intended straight course.

### SUMMARY OF INVENTION

### Technical Problem

Significant force is required to push the existing chisel nail splitting scalpel forward to split the hard toenail. Because the nail plate is thin, and the chisel blade is narrow, the blade can easily slip above or below the nail plate being split. If the chisel blade slips beneath the nail, it gouges deep into the nail bed, causing damage to the tissue, excessive bleeding and postoperative pain for the patient. If the chisel blade slips above the nail, it may gouge the skin on top of the toe, again causing damage, excessive bleeding and postoperative pain, as well as an unnecessary scar.

### Solution to Problem

This invention provides a self centering concave cutting edge on the distal end of the elongated blade. When the blade is pushed forward, splitting the toenail, the sides and deepest part of the concavity in the blade do the cutting because the nail naturally slides to the center of the concavity as the path of least resistance. The nail is effectively sliced above and below, and wedged into the center of the concavity by the peripheral extensions or points of the concave cutting edge of the blade.

### Advantageous Effects of Invention

The concave shape of the cutting edge allows the nail to be split without the tendency for the blade to slip above or below the nail plate. The concave shape also has a sharpened blade above, and below the nail plate, which contact the top and bottom surfaces at an oblique angle. This arrangement of cutting surfaces effectively slices the blade from above and below simultaneously, much as a partially opened scissors cuts a piece of paper when pushed along. Because of this simultaneous oblique slicing effect above and below the nail plate, significantly less force is required to push the scalpel along, splitting the nail plate.

This reduces the manual effort and dexterity required for successful completion of the procedure. Because less manual force is involved the procedure is more controllable, as well as minimizing the risk of slipping, and causing additional injury to the patient or operating surgeon.

### BRIEF DESCRIPTION OF DRAWINGS

FIG. 1 is an oblique perspective view of the nail splitting scalpel in use, the concave sharpened blade centering the scalpel blade on the nail plate and showing the upper distal corner extension above the nail plate.

FIG. 2a is an oblique perspective view of another embodiment of the nail splitting scalpel wherein the concave sharpened blade is rounded and “U” shaped.

FIG. 2b is a side view of FIG. 2a, illustrating the detail of the concave sharpened blade with the distal corner extensions.

FIG. 3a is an oblique perspective view of an embodiment of the nail splitting scalpel wherein the concave sharpened blade is angular and “V” shaped.

FIG. 3b is a side view of FIG. 3a, illustrating the detail of the concave sharpened blade with the distal corner extensions.

### DESCRIPTION OF EMBODIMENTS

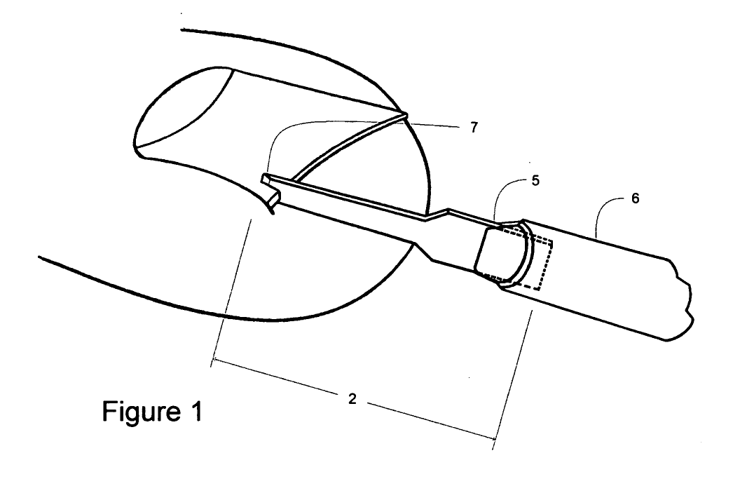

The scalpel blade (see FIG. 1-3a) has an elongated, flat, rectangular shape having a length 2 of about 25-40 mm, and a width 3 of 1.5-4 mm, with a thickness 4 of 0.5-1 mm. It is made of a carbon steel or stainless steel alloy with sufficient hardness to cut through human nail tissue. One end of the scalpel blade is releasably attached 5 to a standard scalpel handle 6, and the other end 7 sharpened into a concave shape. In another embodiment of the invention, the blade and handle are made unitary, and designed to be inseparable.

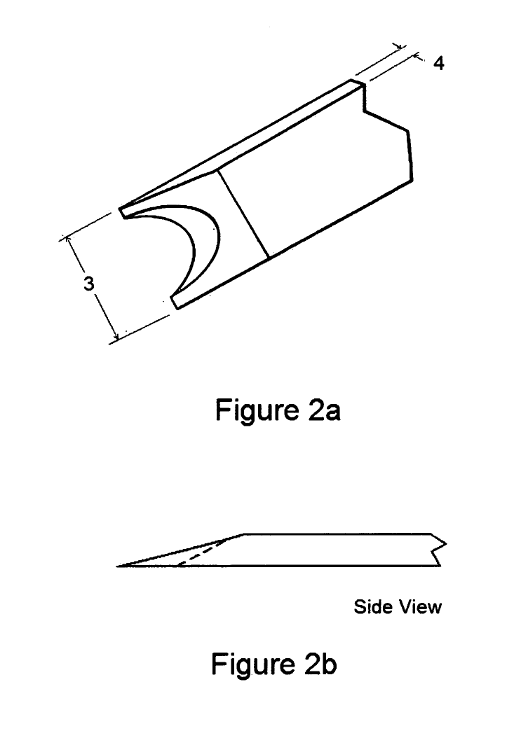

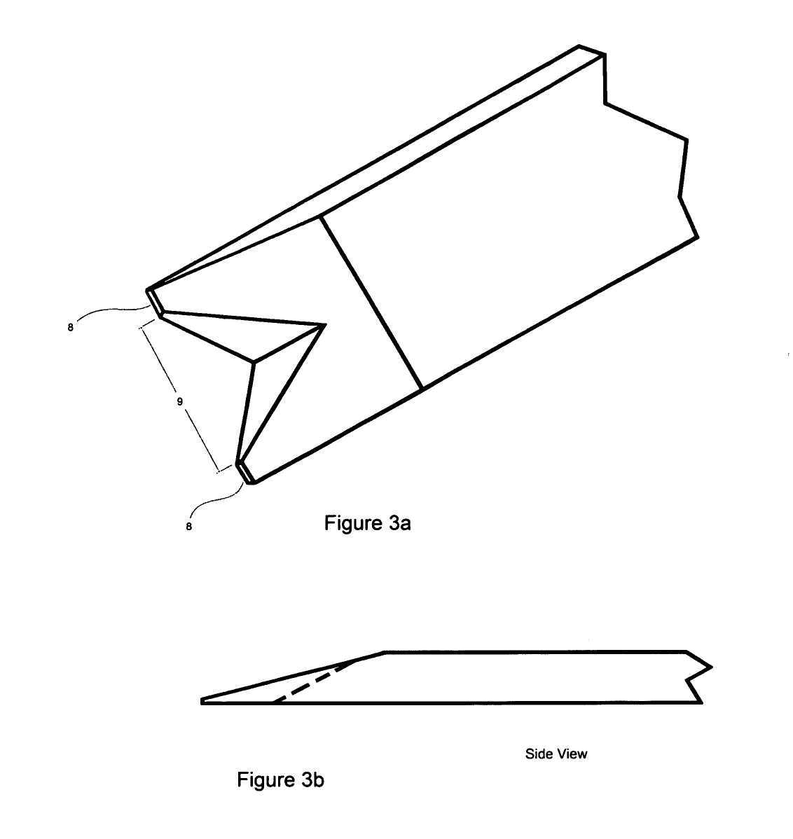

The concave shape may be variously shaped, including rounded a “U” shape shown in FIG. 2a or an angular “V” shape shown in FIG. 3a, with the consistent feature of the concave shape being that the distal corner extensions 8 serve as guides to automatically center the nail plate in the concave sharpened cutting edge 9 as the scalpel blade is pushed forward, splitting the nail plate. The distal corner extensions 8 may be blunt as they do not cut the nail plate.

When the scalpel blade is pushed forward the thin edge of the nail plate slides into the deepest portion of the concavity as the path of least resistance. As the nail plate is split, it tends to keep self centering into the deepest part of the concavity, and is sliced obliquely from above and below, where the sharpened cutting edges slice the nail. The surgeon only needs to guide the scalpel blade in the direction he wishes to split the nail, and apply sufficient force to do so. He doesn't need to worry about the cutting edge slipping above or below the thin nail plate, and because less force is required he has enhanced subtle control.

It requires significant force to push a sharp scalpel blade through hard healthy toenail plate. With the existing art of a straight chisel shaped edge, and the force necessary, it is easy for the blade to slip above or below the nail plate. When the blade slips off the edge of the nail plate, because of the force pushing the scalpel, the blade lurches uncontrollably forward. If it slips below the nail plate, it gouges the vascular and very sensitive nail bed, creating excessive damage which contributes to increased postoperative pain and bleeding. If it slips above the nail plate, it gouges the skin on top of the toe, causing pain, bleeding, and an unsightly scar. If the surgeon is not careful, since he is stabilising the toe with the other hand, if the blade slips, he may accidently cut himself.

With the existing art of a straight chisel shaped edge, to make this procedure easier and safer, many surgeons choose a wider scalpel than necessary, because there is more room for error in keeping the straight chisel shaped edge splitting the nail plate without slipping above or below the nail plate. However, with a chisel shaped blade that is much wider than the thickness of the nail plate, unnecessary damage is done to the nail bed and/or the skin on top of the toe, with the sequelae previously described.

Because the invention slices the nail obliquely from above and below the nail plate as the self centering scalpel is pushed forward, less force is required to split the nail, making for a more controllable and safer nail splitting operation.

The self centering nature of the concave sharpened blade in the invention allows the concave blade to be minimally wider than the thickness of the nail plate, with the distal corner extensions just slightly above and below the nail plate. The selection of this narrow, self centering blade prevents unnecessary damage to the nail bed beneath and the skin above the nail plate where it enters the toe. There is also less chance of the surgeon slipping, and cutting himself.

Because nail plates vary in thickness, usually as a result of metabolic disease or mycotic infection, the concave sharpened blade can be made in varying widths to accommodate the varying thicknesses of nail plates. In this way the surgeon can select a blade that is just wide enough for the thickness of the nail, with the distal corner extensions just slightly above and below the nail plate, and therefore minimise damage to the nail bed or skin.

Numerous modifications may be made to this invention without departing from its scope as defined in the appended claims.

### CITATION LIST

### Patent Literature

| | | |
|---|---|---|
| 1,192,748 | July 1916 | Bundy |
| 2,610,399 | September 1952 | Adams et al. |
| 2,676,595 | April 1954 | Dyekjaer |
| D230007 | January 1974 | Cramer |
| 3,803,713 | April 1974 | Jones et al. |
| 3,835,536 | September 1974 | Marcoux |
| 3,877,147 | April 1975 | Cummings |
| 5,285,577 | February 1994 | Carney et al. |
| 5,341,822 | August 1994 | Farr et al. |
| D353316 | December 1994 | Schmidt et al. |
| 5,524,348 | June 1996 | Tipp |
| 6,182,364 | February 2001 | Reyburn |
| 7,818,885 | October 2010 | Lafauci et al. |
| D481609 | November 2003 | Perlmutter et al. |
| D514916 | February 2006 | Craig |

1. A scalpel blade for splitting toenails, with the cutting edge on the distal end only, in a concave shape with distal corners of the concave shape that serve to guide the toenail plate being split into the center of the concave shaped blade, where it is sliced obliquely from above and below the nail plate as it is pushed forward splitting the toenail plate.

2. The scalpel of claim 1 wherein said blade is designed to be attached to any of various extant scalpel handles (See FIG. 1).

3. The scalpel of claim 1 wherein said blade and handle are unitary and not designed to be separated.

4. The scalpel of claim 1 wherein one of the guiding distal corners extends distal to the other guiding distal corner.

5. The scalpel of claim 4 wherein said blade is designed to be attached to any of various extant scalpel handles.

6. The scalpel of claim 4 wherein said blade and handle are unitary and not designed to be separated.
